(19) United States
(12) Patent Application Publication
(10) Pub. No.: US 2024/0190932 A1
(43) Pub. Date: Jun. 13, 2024

(54) SSX POLYPEPTIDE AND USE THEREOF IN TREATMENT OF SYNOVIAL SARCOMA

(71) Applicant: The Seventh Affiliated Hospital, Sun Yat-Sen University, Shenzhen (CN)

(72) Inventors: Ying ZHANG, Shenzhen (CN); Huaixiang ZHOU, Shenzhen (CN); Difei XU, Shenzhen (CN)

(73) Assignee: The Seventh Affiliated Hospital, Sun Yat-Sen University, Shenzhen (CN)

(21) Appl. No.: 18/537,824

(22) Filed: Dec. 13, 2023

(30) Foreign Application Priority Data
Dec. 13, 2022 (CN) 202211625353.1

(51) Int. Cl. C07K 14/47; A61P 35/00

(52) U.S. Cl. CPC C07K 14/4748; A61P 35/00

(57) ABSTRACT

An SSX polypeptide and use thereof in the treatment of synovial sarcoma are provided. The nucleotide sequence of the SSX polypeptide is set forth in SEQ ID NO: 1. The present invention constructs a plasmid of the SSX polypeptide and expresses it into a polypeptide in synovial sarcoma cells. The polypeptide can effectively inhibit the proliferation of the synovial sarcoma cells and significantly inhibit the expression of oncogenes sox2 and c-myc in the synovial sarcoma cells.

### CROSS REFERENCE TO THE RELATED APPLICATIONS

This application is based upon and claims priority to Chinese Patent Application No. 202211625353.1, filed on Dec. 13, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC160_20231206_seq.xml, created on 12/06/2023, and is 9,299 bytes in size.

### TECHNICAL FIELD

The present invention relates to the technical field of biopharmaceuticals, and in particular to an SSX polypeptide and use thereof in the treatment of synovial sarcoma.

### BACKGROUND

Synovial sarcoma is a type of highly malignant soft tissue sarcoma, which mostly originates from joints, synovium, or soft tissue, hence the name, and rarely occurs in throat, lungs, kidneys, basis cranii, heart, and other sites. The disease mostly occurs in young people of 20-40 years old, and the incidence of the disease accounts for 8%-10% of all soft tissue malignant tumors. The disease has a variety of different subtypes, and the main clinical symptoms of the disease are local swelling, pain, limited activities, and the like. Synovial sarcoma is generally insensitive to radiotherapy and chemotherapy. At present, surgical treatment is mainly used. However, synovial sarcoma is difficult to be eradicated by surgical resection, the local recurrence rate is as high as 50% within two years, and synovial sarcoma easily metastasizes to lungs, bones, lymph nodes, and other sites with poor prognosis. The 5-year survival rate of patients with synovial sarcoma is 20%-50%, and the treatment difficulty is high.

The diagnosis of synovial sarcoma is often delayed due to its insidious onset and slow progression. The image results of traditional imaging examinations such as X-ray examination, arteriography, MRI, and the like have certain reference significance. However, because the peripheral lesions of synovial sarcoma are usually small and it is difficult to distinguish them from some benign lesion areas, missed diagnosis and misdiagnosis are easy to cause, and comprehensive determination needs to be combined with biopsy pathological analysis. Studies have found that the event that SS18 protein is mutated into SS18-SSX definitely induces synovial sarcoma. In recent years, the method for detecting SS18-SSX fusion gene by using fluorescence in situ hybridization or reverse transcription-polymerase chain reaction (RT-PCR) amplification technology has become a widely accepted medical molecular diagnostic basis in the art.

The SS18-SSX mutation is derived from a marked chromatin translocation mutation t (X:18), which usually occurs between an ss18 gene on autosome 18 (SS18: chromosome 18 associated with synovial sarcoma) and three genes on X chromosome (ssx1 gene, ssx2 gene, and rare ssx4 gene), so that 8 amino acids at the C-terminus of the original SS18 protein are substituted with 78 amino acids at the C-terminus of the SSX protein, thereby producing a fusion protein SS18-SSX. This translocation mutation is a key event leading to the development of synovial sarcoma.

Regarding the specific mechanism of how the mutation of the fusion protein SS18 protein into SS18-SSX leads to synovial sarcoma, in 2013, Kadoch and Crabtree at Stanford University School of Medicine published a breakthrough discovery in Cell: SS18 is one of the stable subunits constituting a chromatin remodeling complex BAF (SWI/SNF), and when the fusion protein SS18-SSX is produced, two subunits originally constituting the SWI/SNF complex, i.e., wild-type SS18 and tumor suppressor protein BAF47, are driven away; meanwhile, the transcription of carcinogens such as SOX2/MYC is activated, and the expression of these carcinogens is usually inhibited by the polycomb repressive complex 2 (PRC2). More importantly, the proliferation of synovial sarcoma cells can be inhibited by reversal experiments of removing the fusion protein SS18-SSX and recovering wild-type SS18 and BAF47. Subsequently, targeted disorder of the chromatin remodeling function of the SWI/SNF complex is considered to be a central driver of the pathogenesis of synovial sarcoma. Recent studies have shown that the oncogenic SWI/SNF complex with SS18-SSX is directed to a divalent chromatin region with both transcriptional activation signal H3K4me3 and transcriptional inhibition signal H3K27me3, and the covalent modification signals on these two histones H3 are the products of MLL/COMPASS complex and PRC2 complex, respectively; under the action of the BAF complex, PRC2 is driven away, the inhibition signal H3K27me3 is removed, and the related gene expression is started. Depending on the distribution of the activation signal H3K4me3 in this region, oncogenes PAX7 and MYC are expressed to different extents, which ultimately results in different subtypes of synovial sarcoma, and the resulting tumor heterogeneity is also one of the major causes of difficulty in treating synovial sarcoma. These studies basically determine the core mechanism of synovial sarcoma is that: the fusion protein SS18-SSX activates carcinogens by binding to the chromatin remodeling complex SWI/SNF, resulting in the development of tumors.

That is, the SWI/SNF complex with SS18-SSX (synovial sarcoma fusion protein SS18-SSX) binds to proto-oncogenes such as Sox2 and c-Myc, and reduces the occupancy of histone-modified H3K27me3 that inhibits gene expression at the gene site, resulting in the activation of proto-oncogenes and thus the initiation of abnormal proliferation of cells.

### SUMMARY

The present invention aims to provide an SSX polypeptide, which is overexpressed in synovial sarcoma cells SW982, competitively binds to promoter regions of Sox2 and c-Myc, prevents SS18-SSX in the synovial sarcoma cells from binding to promoters of Sox2 and c-Myc, and down-regulates the transcription levels of the proto-oncogenes Sox2 and c-Myc, so as to inhibit the abnormal proliferation of the cells.

In order to achieve the above objective, the present invention provides the following technical solutions:

The present invention provides an SSX polypeptide, a nucleotide sequence of which is set forth in SEQ ID NO: 1.

The present invention further provides a plasmid overexpressing the SSX polypeptide.

Preferably, a vector of the plasmid is a GV348 vector.

The present invention further provides a cell overexpressing the SSX polypeptide.

Preferably, the cell is a SW982 cell.

The present invention further provides use of the polypeptide, the plasmid, or the cell in the manufacture of a reagent for inhibiting oncogenes sox2 and/or c-myc.

The present invention further provides use of the polypeptide, the plasmid, or the cell in the manufacture of a medicament for treating synovial sarcoma.

Since SS18-SSX directs chromatin remodeling complex BAF to be mislocated on the gene nucleic acid, the expression of the proto-oncogenes (e.g., SOX2 and C-MYC genes) is activated, thereby inducing synovial sarcoma. Therefore, we intended to reverse the progression of synovial sarcoma by adding excess SSX and then inhibiting SS18-SSX functions through competitive binding. The hypothesis model diagram is shown in FIG. 1.

The present invention constructs a plasmid of the SSX polypeptide and expresses it into a polypeptide in the synovial sarcoma cells. The polypeptide can effectively inhibit the proliferation of the synovial sarcoma cells and significantly inhibit the expression of the oncogenes sox2 and c-myc in the synovial sarcoma cells. It is shown that the SSX polypeptide is expected to become a simple, safe and effective novel method for preventing and treating synovial sarcoma.

### BRIEF DESCRIPTION OF THE DRAWINGS

FIG. 1 is a hypothesis model diagram showing the inhibition of SS18-SSX by SSX polypeptide;

FIG. 2 shows the transcription levels of Sox2 and c-Myc genes before and after SSX overexpression detected in the RT-qPCR experiment in Example 2; **P<0.01 and ***p<0.001;

FIG. 3 shows the proliferative ability of synovial sarcoma cells SW982 before and after SSX overexpression detected in the cck8 cell proliferation experiment in Example 3; *p<0.05; and

FIG. 4 shows the proliferative ability of the synovial sarcoma cells SW982 before and after SSX overexpression detected in the colony formation experiment in Example 4; *p<0.05.

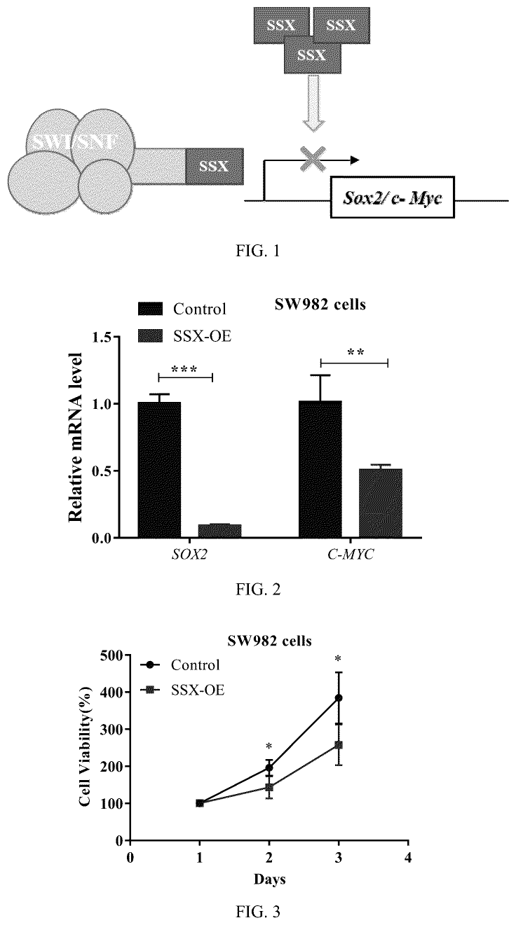

### DETAILED DESCRIPTION OF THE EMBODIMENTS

The technical solutions provided by the present invention will be described in detail below with reference to the examples, which, however, should not be construed as limiting the protection scope of the present invention.

### Example 1

### 1. Construction of Plasmid

Taking cDNA of synovial sarcoma cells as a template, PCR amplification was performed by using a primer 1 and a primer 2 to obtain an SSX gene sequence set forth in SEQ ID NO: 1.

Primer 1: ccaactttgtgccaaccggtcgccaccatggactacaaggatgacgatgacaag, as shown in SEQ ID NO: 2; and

Primer 2: cacacattccacaggaattttattcgtcatcctcttccggatc, as shown in SEQ ID NO: 3.

The SSX polypeptide sequence was constructed on a vector GV348 (purchased from Shanghai GeneChem Co., Ltd.) through Agel/EcoRI enzyme digestion to give a GV348-SSX plasmid.

### 2. Plasmid Transfection and Lentivirus Harvesting

24 h before transfection, 293T cells in the logarithmic growth phase were digested with trypsin, the cell density was adjusted to 5×10⁶ cells/15 mL with a medium containing 10% serum, and the cells were re-seeded in a 10 cm cell culture dish and incubated in an incubator at 37° C. with 5% CO₂ for 24 h. When the cell density reached 70%-80%, the cells could be used for transfection. The medium was replaced with a serum-free medium 2 h before transfection.

1 mL of OPTI-MEM medium and each DNA solution (20 μg of the GV348-SSX vector plasmid, 15 μg of a pHelper 1.0 vector plasmid, and 10 μg of a pHelper 2.0 vector plasmid) were added to a sterilized centrifuge tube. After the mixture was shaken and mixed well, 100 μL of PEI 40K was added, the mixture was shaken, mixed well, and incubated at room temperature for 15 min.

The mixed solution was slowly added dropwise to the culture solution of the 293T cells, mixed well, and incubated in the cell incubator at 37° C. with 5% CO₂. (Note: the addition process must be uniform, and the cells should not be pipetted as much as possible.) After 6 h of incubation, the medium containing the transfection mixture was discarded, and 10 mL of PBS was added for washing once. The culture dish was gently shaken to wash the residual transfection mixture, and then the residual transfection mixture was discarded. 20 mL of a cell culture medium containing 10% serum was slowly added, and the cells were incubated in the incubator at 37° ° C. with 5% CO₂ for another 48 h to give 293T cells transfected with the GV348-SSX plasmid.

### 3. Lentivirus Concentration and Purification

According to the cell state, the 293T cell supernatants 48 h after transfection (counted as 0 h when transfection) were collected and centrifuged at 4000 g at 4° C. for 10 min to remove cell debris. The supernatants were filtered with a 0.45 μm filter in 40 mL ultracentrifuge tubes, and the samples were balanced. The ultracentrifuge tubes containing the virus supernatants were put into a Beckman ultracentrifuge one by one, the centrifugation parameter was set to 25000 rpm, the centrifugation time was 2 h, and the centrifugation temperature was controlled at 4° C. After the centrifugation, the supernatants were discarded, and the liquid remaining on the tube wall was removed as much as possible. A virus preservation solution (PBS) was added thereto and gently and repeatedly pipetted to resuspend the cells. (Note: there was a certain degree of loss of virus recovery in this step, avoiding as much as possible prolonged exposure of the virus to room temperature.)

After complete dissolution, the samples were centrifuged at a high speed of 10000 rpm for 5 min, and then the supernatants were collected and aliquoted to give the concentrated and purified lentiviruses.

### 4. Lentivirus Transfection

SW982 cells in good state were seeded on a 24-well plate to the cell confluence of 50%. The cells were incubated at 37° ° C. overnight. Before infection, the lentiviruses were taken from a refrigerator and rapidly thawed in a 37° C. water bath. The virus stock solution was added to the cells (MOI=3) and gently mixed well. 12 h after infection, the virus-containing medium was pipetted off and replaced with a fresh complete culture solution. The cells were continuously incubated at 37° C. 48 h after infection, 2 μg/mL of puromycin was added for screening, and the solution was replaced with a complete culture solution containing puromycin once every 2 days until cells in a non-infection screening control group were killed by puromycin. The cells were continuously screened and passaged 3 times to give an SW982 stable cell strain overexpressed SSX. The stable cell strain was cryopreserved.

The multiplicity of infection (MOI) refers to the number of viruses infected per cell, and generally, the higher the MOI, the higher the number of chromosomes that the viruses integrated into and the expression amount of target proteins.

### Example 2

### 1. RNA Extraction

1 mL of TRIzol was added to the SW982 cell culture plate in which the SW982 cells were SSX-overexpressed to lyse the cells. The mixture was pipetted several times using a pipette. The plate was left at room temperature (25° C.) for 5 min to completely separate the nucleic acid-protein complex.

0.2 mL of chloroform was added, followed by vigorous shaking for 15 s. The plate was left at room temperature for 3 min and centrifuged at 10000×g at 4° C. for 15 min. The sample was divided into three layers, that is, a bottom layer of a yellow organic phase, an upper layer of a colorless aqueous phase, and an intermediate layer. RNA was mainly in the aqueous phase, the volume of which was 60% of that of the TRIzol reagent used.

The aqueous phase was transferred to a new tube, and RNA in the aqueous phase was precipitated with isopropanol (in a volume ratio of 0.5:1 to TRIzol). The tube was left at room temperature for 10 min and centrifuged at 10000×g at 4° C. for 10 min. No RNA precipitate was observed before centrifugation, and a gelatinous precipitate appeared on the tube side and the tube bottom after centrifugation. The supernatant was removed.

The RNA precipitate was washed with 75% ethanol (in a volume ratio of 1:1 to TRIzol). The tube was centrifuged at no more than 7500×g at 4° C. for 5 min to discard the supernatant.

The RNA precipitate was evacuated to dryness in vacuum and let stand for 5 min (do not centrifuge and dry in vacuum; excessive drying would result in greatly reduced solubility of RNA). 100 μL of RNase-free water was added, and the mixture was pipetted several times using a pipette tip and left at 60° C. for 10 min to dissolve the RNA. The mixed solution was preserved at −70° C.

2. Synthesis of cDNA by Reverse Transcription of RNA

The RNA extracted as described above was reversely transcribed into cDNA with reference to the instruction of FastStart Universal SYBR Green Master (ROX) (Roche). Specifically, RT reaction solutions were prepared as per Table 1 (the preparation was performed on ice). In order to ensure the accuracy of the preparation of the reaction solutions and reduce errors caused by aliquoting, the reaction solutions were prepared according to volumes slightly larger than the actual amounts, and the RNA sample was added finally.

TABLE 1
RT reaction solutions

| | | |
|---|---|---|
| 5× PrimerScript Buffer (for Real Time) | 2 | μL |
| PrimerScript RT Enzyme Mix I | 0.5 | μL |
| Total RNA | 500 | ng |

| | |
|---|---|
| RNase & DNase Free dH₂O | up to 10 μL |

After the addition of the above reagents, the mixture was instantaneously centrifuged at a rotation speed of 13000 rpm, incubated in a water bath at 50° C. for 15 min, and then incubated in a water bath at 85 ºC for 5 s. The mixture was placed on ice for 3 min and preserved at −20° C. for later use.

### 3. Fluorescent Quantitative PCR Analysis

The system was amplified using ABI Prism 7300 Real-Time PCR Systems with reference to the instruction of PrimeScript™RT reagent Kit (Takara) according to the following reaction system. 5.8SrRNA of Gastrodiae Rhizoma was used as an internal reference. The experiment was repeated three times, and three replicates were made for each sample.

TABLE 2
Standard sample reaction system

| | | |
|---|---|---|
| SYBR Green I Mix | 12.5 | μL |
| Forward primer | 0.5 | μL |
| Reversed primer | 0.5 | μL |
| cDNA | 1 | μL |
| RNase & DNase Free dH₂O | 10.5 | μL |
| Total volume | 25 | μL |

The system was mixed well and aliquoted into a 96-well optical plate. The plate was simply centrifuged and loaded on a machine. The amplification procedure was: 95° C. for 10 min; 95° C. for 15 s, 40 cycles; 60° C. for 1 min; 95° C. for 15 s, 2 cycles; 60° C. for 1 min. The qPCR primers are shown in Table 3.

TABLE 3
qPCR primers

| | Forward primer | Reversed primer |
|---|---|---|
| Gene | (5′-3′) | (5′-3′) |
| sox2 | tacagcatgtcctactcgcag | gaggaagaggtaaccacaggg |
| | (SEQ ID NO: 4) | (SEQ ID NO: 5) |
| c-myc | gtcaagaggcgaacacacaac | ttggacggacaggatgtatgc |
| | (SEQ ID NO: 6) | (SEQ ID NO: 7) |
| ß- | catgtacgttgctatccaggc | ctccttaatgtcacgcacgat |
| actin | (SEQ ID NO: 8) | (SEQ ID NO: 9) |

The results are shown in FIG. 2. FIG. 2 shows SSX overexpressed in the synovial sarcoma cells SW982 can competitively bind to promoter regions of Sox2 and c-Myc, prevent SS18-SSX in the synovial sarcoma cells from binding to promoters of Sox2 and c-Myc, and down-regulate the transcription levels of the Sox2 and c-Myc genes, so as to inhibit the abnormal proliferation of the cells.

### 4. Processing of Fluorescent Quantitative PCR Data

The CT value, amplification curve, and dissolution curve analyses were performed on the fluorescent quantitative PCR results by ABI Prism 7500 SDS Software. The CT value referred to the number of cycles required for the fluorescence signal in the reaction tube to reach the set value in the experiment in the reaction system; there was a linear relationship between the CT value of the target gene and the logarithm of the initial copy number of the target gene, and the CT value was inversely proportional to the initial copy number. The dissolution curve was used for verifying the existence of non-specific amplification; if a single peak value appeared, it indicated that the product had relatively good specificity, and otherwise, the product was non-specific.

Relative quantification referred to the change in the amount of a target gene in a sample relative to a reference sample, and was concerned with how many times the transcription level of the target gene changed, rather than how much it was transcribed. Common methods for calculating the relative quantification were comparative 2^{-ΔΔCT} method, double-standard curve method, PFAFF method, and the like. In this experiment, the analysis results were processed using Excel software, and the expression difference of mature miRNA was analyzed using the 2^{-ΔΔCT} method, that is, ΔΔC_{T}=ΔC_{T sample}−ΔC_{T control}, and ΔC_{T}=C_{T target gene}−C_{T internal reference gene}.

### Example 3

SW982 cells and SSX-overexpressed SW982 cells were made into 1×10⁴ cells/mL, and the cells were added to a 96-well plate containing a culture solution at 100 μL per well (i.e., 1×10³ cells). On day 1, day 2, and day 3 of the culture, the culture solution was discarded, and a medium containing 10% CCK8 was added in a buffer-exchanging manner. In order to avoid the formation of air bubbles during the addition, the pipette tip was immersed in the culture solution to slowly add the medium. The plate to which CCK-8 was added completely was incubated in an incubator at 37° C. with 5% CO₂ for 2 h and stained in the incubator for 2 h. The absorbance value was measured at a wavelength of 570 nm on a microplate reader. The data were analyzed and processed to plot the proliferation curve.

Cell proliferation rate %=(cell OD on day×/cell OD on day 1)×100%.

The results are shown in FIG. 3, indicating that the proliferation ability of the synovial sarcoma cells SW982 after SSX overexpression was significantly inhibited in the cck8 cell proliferation experiment.

### Example 4

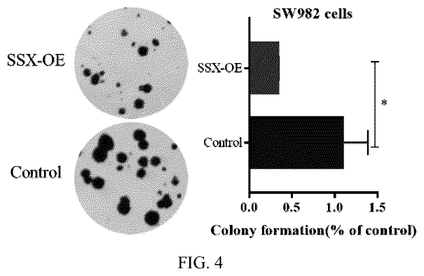

SW982 cells and SSX-overexpressed SW982 cells were seeded into 6-well plates at 500 cells/well, the clones grew for 14 days, the supernatants were pipetted off, and the remaining cells were gently washed twice with PBS; 4% paraformaldehyde solution was added at room temperature, and the mixtures were treated for 15 min to fully immobilize the cells; after the 4% paraformaldehyde solution was discarded, a crystal violet staining solution was added to treat the cells for 15 min; then, a small amount of PBS solution was added, the unbound redundant staining solution was washed away, and the cells were naturally dried. Photographic records were made as shown in FIG. 4.

As can be seen from FIG. 4, the proliferation ability of the synovial sarcoma cells SW982 overexpressing SSX was significantly inhibited in the colony formation experiment.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A method of manufacturing a medicament for treating synovial sarcoma, comprising using an SSX polypeptide, wherein a nucleotide sequence encoding the SSX polypeptide is set forth in SEQ ID NO: 1.

2. A method of manufacturing a medicament for treating synovial sarcoma, comprising using a plasmid overexpressing an SSX polypeptide, wherein a nucleotide sequence encoding the SSX polypeptide is set forth in SEQ ID NO: 1.

3. The method according to claim 2, wherein a vector of the plasmid is a GV348 vector.

4. A method of manufacturing a medicament for treating synovial sarcoma, comprising using a cell overexpressing an SSX polypeptide, wherein a nucleotide sequence encoding the SSX polypeptide is set forth in SEQ ID NO: 1.

5. The method according to claim 4, wherein the cell is a SW982 cell.


SEQUENCE LISTING (9 sequences)

SEQ ID NO: 1 - DNA, 240 bp, synthetic construct
  source 1..240: mol_type = other DNA
atgattatgccgaaaaaaccggcggaagatgaaaacgacagcaaaggcgtgagtgaagccagcggtccgcaaaacgatggcaagcagctgcatccgccaggtaaagccaatatcagtgagaaaatcaacaaacgcagcggcccgaaacgcggcaaacatgcctggacgcatcgcctgcgtgaacgtaaacaactggtgatttatgaagaaatcagcgatccggaagaggatgacgaataa

SEQ ID NO: 2 - DNA, 54 bp, synthetic construct
  source 1..54: mol_type = other DNA
ccaactttgtgccaaccggtcgccaccatggactacaaggatgacgatgacaag

SEQ ID NO: 3 - DNA, 43 bp, synthetic construct
  source 1..43: mol_type = other DNA
cacacattccacaggaattttattcgtcatcctcttccggatc

SEQ ID NO: 4 - DNA, 21 bp, synthetic construct
  source 1..21: mol_type = other DNA
tacagcatgtcctactcgcag

SEQ ID NO: 5 - DNA, 21 bp, synthetic construct
  source 1..21: mol_type = other DNA
gaggaagaggtaaccacaggg

SEQ ID NO: 6 - DNA, 21 bp, synthetic construct
  source 1..21: mol_type = other DNA
gtcaagaggcgaacacacaac

SEQ ID NO: 7 - DNA, 21 bp, synthetic construct
  source 1..21: mol_type = other DNA
ttggacggacaggatgtatgc

SEQ ID NO: 8 - DNA, 21 bp, synthetic construct
  source 1..21: mol_type = other DNA
catgtacgttgctatccaggc

SEQ ID NO: 9 - DNA, 21 bp, synthetic construct
  source 1..21: mol_type = other DNA
ctccttaatgtcacgcacgat